(19) United States
(12) Patent Application Publication
(10) Pub. No.: US 2024/0024136 A1
(43) Pub. Date: Jan. 25, 2024

(54) MEDICAL KIT FOR TREATMENT OF VASCULAR DISEASES

(71) Applicant: Acandis GmbH, Pforzheim (DE)

(72) Inventors: Andreas Schüssler, Pfinztal (DE); Andrés Braschkat, Bretten (DE); Andreas Ding, Karlsruhe (DE)

(21) Appl. No.: 18/037,528

(22) PCT Filed: Nov. 19, 2021
(86) PCT No.: PCT/EP2021/082253

§ 371 Date: May 17, 2023

(30) Foreign Application Priority Data
Nov. 26, 2020 (DE) 202020106807.5

(51) Int. Cl. A61F 2/90; A61F 2/966

(52) U.S. Cl. CPC A61F 2/90; A61F 2/966; A61F 2002/9665; A61F 2250/0098

(57) ABSTRACT

The disclosure relates to a medical kit for treating vascular diseases, including a tubular or sleeve-type insertion aid, a transport wire having a distal tip, and a stent having a wire mesh which consists of a plurality of wires that, at a proximal stent end define loops and, at a distal stent end, open wire ends. The stent is arranged on the transport wire and together with the transport wire inside the insertion aid. The distal stent end is closer to the distal tip of the transport wire than the proximal stent end.

The invention relates to a medical kit for the treatment of diseases of blood vessels.

Currently, stents for the treatment of diseases of blood vessels such as stenoses or aneurysms are known which are provided in a kit with an introductory aid, also known as introducer, and a transport wire. The stent is usually disposed on the transport wire in a compressed state and positioned with the transport wire in the introducer. The introducer essentially forms a tube or shaft, which maintains the stent in the compressed state.

For the actual treatment, the stent with the transport wire is guided through a catheter to the treatment site. At the treatment site, the stent is released from the catheter and deploys there. In some treatment methods, after complete release of the stent into the blood vessel, it may be necessary to guide the catheter through the stent in the distal direction. This may be necessary, for example, in order to expand the stent again actively by means of a balloon or to press it against the blood vessel wall. Other reasons for introducing the catheter into the expanded stent include when a region of the blood vessel which is disposed distally from the stent has to undergo additional treatment. Sometimes, operators also guide the catheter through the expanded stent in order to ensure that the stent is sufficiently accessible.

In this procedure, in which the catheter is pushed distally and is guided through the stent, there is a risk that the catheter will become caught on the wire ends of the stent. Depending on the construction of the stent, it may in particular be the case that the proximal stent end does not widen sufficiently, so that wire ends can readily protrude into the blood vessel. In this case, when the catheter is pushed distally, the catheter tip can become caught on these wire ends. This can lead to the stent being released further from the blood vessel wall, or the blood vessel wall being damaged due to a pushing movement acting on the stent. Furthermore, there is a risk that the catheter cannot be pushed through the stent so that, for example, a treatment of downstream regions of the blood vessel using the catheter is no longer possible.

There has been no recognition of the problem described above in the prior art. S tents are in fact known, which are formed from wires that form loops at at least one end of the stent. Stents of this type have been described, for example, in DE 10 2012 112 730 A1 and DE 10 2015 107 291 A1, but no indication is given therein as to how catching of a catheter tip on the wire ends of a proximal end of the respective stent could be prevented.

DE 10 2018 333 345 A1 describes a stent which is formed from a single wire that is formed into loops at both ends of the stent. The known stent is characterized by a particular expansion behaviour. The procedure described above in which the catheter is pushed distally through the expanded stent and the problem associated therewith was not recognised, however.

DE 20 2013 012 692 U1 in fact discloses a stent which may be formed from a plurality of individual wires, wherein the individual wires are soldered, bonded or fused together at one or both ends into shaped loops. However, it is not clear therefrom which configuration of the stent is to be used for which function. In addition, not only is the manufacture of the loops complicated, but connecting the individual wires together, in particular at the highly stressed ends of the stents, entails the risk that the connection points could break and produce open wire ends which could lead to damage to the vessel walls.

Thus, the objective of the invention is to provide a medical kit for the treatment of diseases of blood vessels which reduces the risk of catching between the catheter tip and a stent which is expanded in the blood vessel and thus enables a subsequent treatment with the catheter.

In accordance with the invention, this objective is achieved by means of the subject matter of claim 1.

Thus, the invention is based on the concept of providing a medical kit for the treatment of diseases of blood vessels wherein the kit is equipped with
a tubular or shaft-like introducer,
a transport wire which has a distal tip, and
a stent comprising a meshwork of multiple wires which form loops at a proximal stent end and which form open wire ends at a distal stent end, wherein the stent is disposed on the transport wire and disposed therewith inside the introducer, and wherein the distal longitudinal end of the stent is positioned closer to the distal tip of the transport wire than the proximal longitudinal end.

The invention makes use of two aspects in order to prevent a catheter tip from catching on the proximal stent end.

On the one hand, in the kit in accordance with the invention, advantageously, the stent has loops at one stent end. The curvature of the loops allows that a catheter tip is deflected and is therefore guided into the interior of the stent. Thus, the catheter tip cannot become caught as easily, as is the case with open wire ends, for example.

On the other hand, the invention explicitly provides that the stent is positioned on the transport wire in a manner such that the loops are disposed at a proximal stent end. Thus, with the kit, the arrangement of the transport wire with the stent in the introducer ensures that the stent can only be introduced into a blood vessel in a manner such that the loops are orientated proximally in the blood vessel. Thus, when they are released from a catheter, the loops flare out last and therefore lie close to the catheter tip. If the catheter is now to be pushed again into the stent, then this generally occurs from the side on which the loops are positioned. In this manner, the risk of the catheter catching on the stent when the catheter is pushed distally into the stent is significantly reduced.

A further advantageous effect of the loops disposed at the proximal stent end is that the loops increase the radial force at the proximal stent end. The two limbs of a loop then in fact tend to move away from each other, i.e. to splay out. The result is that the proximal stent end flares out or expands very well. This means that the risk of wires protruding into the blood vessel at the proximal end of the stent because they do not bear against the vessel wall sufficiently well is also reduced.

In summary, all of the aforementioned effects mean that after complete release of the stent, a catheter can readily be pushed distally through the stent, whereupon the danger of catching between the catheter and the stent is significantly reduced.

In order to be able to detect the proximal stent end upon implantation of the stent in a blood vessel, in a preferred embodiment of the invention, the proximal stent end, in particular the loops, is disposed between a proximal and a distal radiographic marker of the transport wire. The radiographic markers are readily discernible under radiographic monitoring, so that the surgeon can detect the current location of the proximal stent end. This is essential in order to be able to assess whether the final position of the stent is correct in the case of partial release of the stent. As soon as the positioning is complete, the stent can then be released fully from the catheter.

Preferably, the transport wire has a transport section and a guide section. The guide section may have a diameter, which is larger than a diameter of the transport section. Furthermore, the guide section may have an abutment surface on which the loops of the proximal stent end bear in the axial direction. The abutment surface therefore enables the stent to be pushed through the introducer and/or the catheter. For this reason, the loops bear on the abutment surface in the axial direction. Preferably, in addition, the guide section of the transport wire has a cross sectional diameter which substantially corresponds to an internal diameter of the introducer and/or of a catheter. This therefore ensures that the stent remains in the transport section adjoining the guide section. In particular, this can therefore prevent the stent from becoming positioned between an inner wall of the introducer and/or of the catheter and the transport wire, which could result in the stent no longer being capable of being transported through the transport wire.

In a preferred variation of the invention, the proximal radiographic marker of the transport wire is disposed at a distal end of the guide section. Furthermore, the proximal radiographic marker may directly border the abutment surface and/or may form the abutment surface. This arrangement of the proximal radiographic marker ensures that a surgeon can readily detect the proximal stent end under radiographic monitoring. In particular, in this manner, the release of the stent into a blood vessel can be controlled particularly well because the surgeon is in possession of information regarding how the proximal end of the stent nears the catheter tip.

Preferably, the transport section of the transport wire extends completely through the stent. The distal tip of the transport wire may be a part of the transport section. Preferably, the distal tip is flexible, in particular more flexible than the remainder of the transport section. Thus, the distal tip, which preferably protrudes beyond the stent, can navigate well through tortuous blood vessels.

In a preferred variation of the invention, the distal radiographic marker of the transport wire is disposed in the transport section. In particular, the distal radiographic marker may be disposed between the distal tip and the abutment surface of the guide section. The distal radiographic marker preferably has an external diameter, which is smaller than the external diameter of the guide section, so that a gap is formed between the distal radiographic marker and the inner wall of the introducer, through which the meshwork of the stent can extend. Preferably, the distal radiographic marker is positioned close to the proximal end of the stent. Particularly advantageously, the loops of the proximal stent end are disposed between the proximal radiographic marker and the distal radiographic marker of the transport wire. In this manner, the position of the loops can be discerned particularly well by a surgeon under radiographic monitoring.

In a preferred variation of the invention, the transport wire, in particular the guide section, may protrude proximally out of the introducer and have a proximal gripping section with ribbing. The design of the gripping section is advantageous because in this manner, the transport wire can be grasped correctly by the surgeon. Good contact with the transport wire is advantageous in order to be able to guide the transport wire properly through a catheter and a blood vessel. The ribbing here prevents slipping by the surgeon along the transport wire. The ribbing in the gripping section improves the navigability of the transport wire.

In a further preferred embodiment of the invention, a distal end of the introducer has a connector for connection to a catheter. Thus, the introducer can be directly connected to a catheter. In this manner, the stent with the transport wire can easily be transferred from the introducer into a catheter. By means of the catheter, the stent, which is also disposed on the transport wire, can be brought into the blood vessel to be treated.

In a further preferred embodiment of the invention, first loops and second loops are disposed at the proximal stent end of the stent. The first loops may protrude beyond the second loops in the axial direction. In particular, the same number of first loops and second loops may be provided at the proximal stent end. Preferably, the first loops and the second loops are disposed in alternation in the circumferential direction.

Thus, in this embodiment of the invention, essentially, alternating longer and shorter loops are disposed at the proximal stent end. In this regard, a longer loop alternates with a respective shorter loop. In the context of the present application, the longer loops are designated as first loops and the shorter loops are designated as second loops. In the kit, the first loops preferably bear against the abutment surface. The staggered arrangement of the loops has the advantage that in this manner, at the proximal stent end as well, the stent can be properly compressed to a small cross sectional diameter. In addition, this substantially facilitates reinsertion of a catheter into the expanded stent. The danger of the catheter becoming caught on the proximal stent end is further reduced thereby.

In order to make it possible for the stent to be visible under radiographic monitoring, preferably, the wires of the meshwork have a core comprising a radiopaque material and a sheath comprising a shape memory material. The radiopaque core enables the entire meshwork to be detected under radiographic monitoring. This means that not only can the position of the meshwork be detected, but also, whether or not the meshwork or the stent is a sufficiently close fit against the vessel wall can be detected. The sheath produced from the shape memory material provides self-expandability. Thus, following release from a catheter, the stent expands automatically and preferably retains the expanded form. Preferably, a nickel-titanium alloy is used as the shape memory material. The radiopaque material for the core may comprise platinum, gold or tantalum. A platinum-iridium alloy is particularly preferred.

In order to ensure a sufficiently high radiopacity, in a further variation of the invention, at least 20% by volume, in particular at least 25% by volume, in particular at least 30% by volume of each wire is formed by the core. Preferably, at most 40% of the wire is formed by the core.

In one embodiment of the invention, the stent has no additional radiographic markers and/or exclusively consists of wires. This prevents the properties of the meshwork from being compromised by additional constructional elements. In particular, radiographic markers, which are crimped onto the wires make feeding a stent through a catheter more difficult, because radiographic markers of that type can rub on the inner wall of the catheter. Dispensing with additional elements of that type, then, improves the feeding capability of the stent.

Alternative embodiments of the invention in which the stent has additional radiographic markers are not excluded, however. As an example, a plurality, in particular three, additional radiographic markers may be disposed at the proximal stent end, in particular at the loops, in order to highlight the proximal stent end under radiographic monitoring.

In a particularly preferred variation of the invention, the stent of the kit has a meshwork of wires with a diameter of 30 μm. Preferably, the meshwork comprises 24 wires, wherein each wire forms a respective loop. Consequently, there are 24 loops provided at the proximal stent end. Because the wires turn round in order to form the loops, there are effectively 48 wire segments over the circumference of the meshwork. Preferably, loops are present at the proximal stent end only. In this preferred variation of the invention, the wires form open wire ends at the distal stent end. Preferably, the meshwork has a cylindrical meshwork section which comprises the distal stent end and which extends to the proximal stent end. Thus, the proximal stent end is no longer part of the cylindrical meshwork section, but the distal stent end is part thereof. In the cylindrical meshwork section, an angle of twist may be set at between 70° and 75°. At the proximal stent end, i.e. at the transition into the loops, the angle of twist may be smaller. In particular, the angle of twist at the proximal stent end may be 60°. The angle of twist is that angle which is formed between a projection of the longitudinal axis of the meshwork onto the plane of the wall in which the wires are interlaced, and one of the wires. Moreover, the proximal stent end may fan out conically, i.e. in the expanded, non-operational state which is not subjected to forces, the loops widen out at the proximal stent end, so that the vertices of the loops are disposed on a cross sectional diameter, which is larger than the cross sectional diameter of the cylindrical meshwork section. In this variation, the stent may in particular have a nominal diameter, i.e. a diameter, which is taken up by the stent in the implanted state, of 3.5 mm or 3.0 mm or 2.5 mm. In the self-expanded non-operational state, which is not subjected to forces, the diameter is preferably somewhat larger (“oversizing”), so that in the implanted state, the stent bears correctly against a vessel wall. Preferably, a stent with a nominal diameter of 2.5 mm has a cross sectional diameter in the non-operational state which is approximately 2.62 mm. In the case of a stent with a nominal diameter of 3.0 mm, the cross sectional diameter in the non-operational state is approximately 3.12 mm and in the case of a stent with a nominal diameter of 3.5 mm, the cross sectional diameter in the non-operational state is approximately 3.62 mm. The cross sectional diameter in the non-operational state here is with respect to the cylindrical meshwork section of the stent.

Particularly preferably, the dimensions of the stent of a kit in accordance with the invention are as follows:

| | | | | | | | | Length of |
|---|---|---|---|---|---|---|---|---|
| | | Number | Wire diameter | | | | Length of | cylindrical |
| | Stent | of loops | d/mm | | | Total | proximal | meshwork |
| | diameter | (wires) | (tolerance | Angle of twist | | length | stent end | section |
| | OD stent/mm | nl | according to | AT-L3/° | AT-L2/° | L1/mm | L2/mm | L3/mm |
| Embodiment | (±0.2 mm) | (nw)/— | specifications) | (±2°) | (±4°) | (±1 mm) | (±1 mm) | (±1 mm) |
| 1 | 2.62 | 24 (48) | 0.03 | 70 | 60 | 8.0 | 3.0 | 5.0 |
| 2 | 2.62 | 24 (48) | 0.03 | 70 | 60 | 12.0 | 3.0 | 9.0 |
| 3 | 2.62 | 24 (48) | 0.03 | 70 | 60 | 15.0 | 3.0 | 12.0 |
| 4 | 2.62 | 24 (48) | 0.03 | 70 | 60 | 19.0 | 3.0 | 16.0 |
| 5 | 3.12 | 24 (48) | 0.03 | 75 | 60 | 8.0 | 3.0 | 5.0 |
| 6 | 3.12 | 24 (48) | 0.03 | 75 | 60 | 11.0 | 3.0 | 8.0 |
| 7 | 3.12 | 24 (48) | 0.03 | 75 | 60 | 14.0 | 3.0 | 11.0 |
| 8 | 3.12 | 24 (48) | 0.03 | 75 | 60 | 18.0 | 3.0 | 15.0 |
| 9 | 3.62 | 24 (48) | 0.03 | 75 | 60 | 8.0 | 3.5 | 4.5 |
| 10 | 3.62 | 24 (48) | 0.03 | 75 | 60 | 12.0 | 3.5 | 8.5 |
| 11 | 3.62 | 24 (48) | 0.03 | 75 | 60 | 15.0 | 3.5 | 11.5 |
| 12 | 3.62 | 24 (48) | 0.03 | 75 | 60 | 19.0 | 3.5 | 15.5 |

The invention will now be described in more detail with the aid of an exemplary embodiment and with reference to the accompanying schematic drawings, in which:

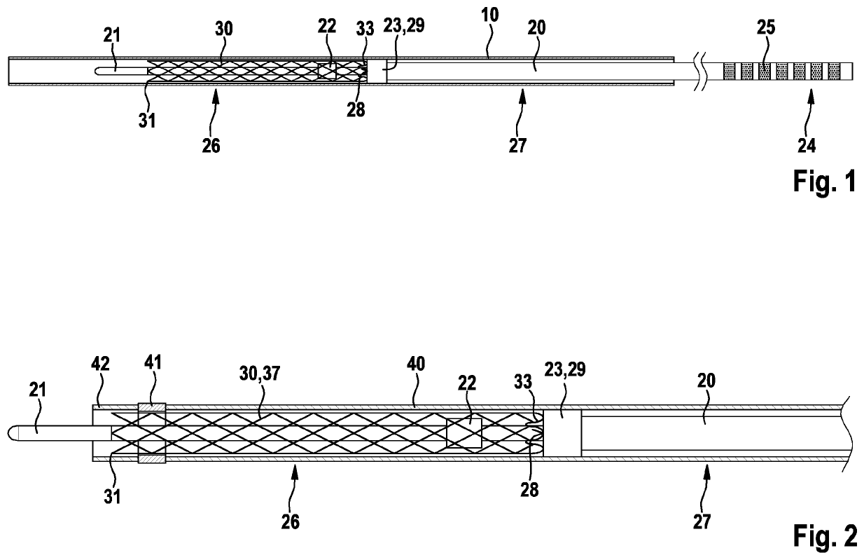

FIG. 1 shows a longitudinal sectional view through a medical kit in accordance with the invention in a preferred exemplary embodiment;

FIG. 2 shows a longitudinal section through a catheter with the transport wire and the stent from the kit in accordance with FIG. 1;

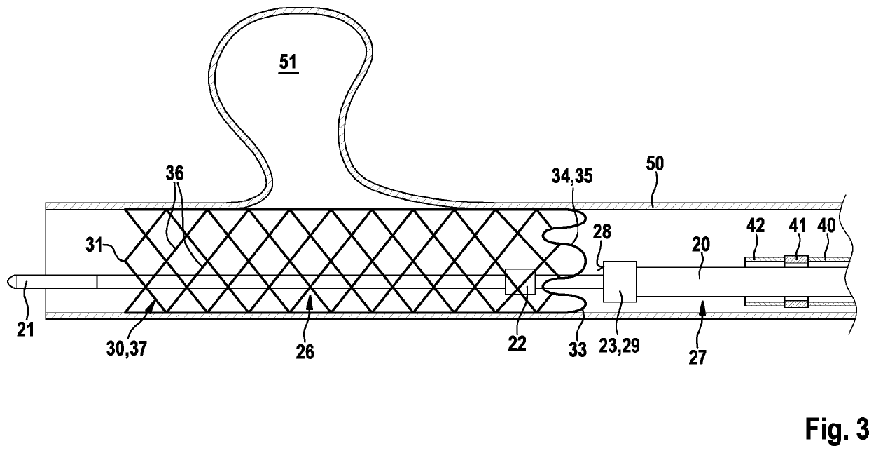

FIG. 3 shows a longitudinal sectional view through a blood vessel when inserting the stent from the kit of FIG. 1;

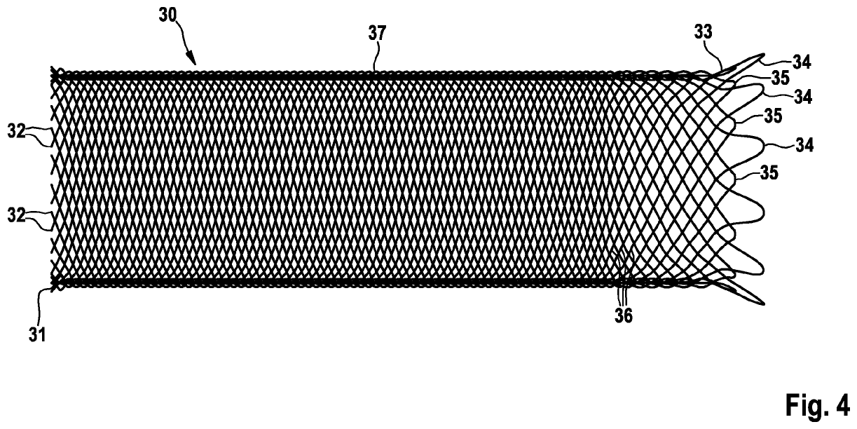

FIG. 4 shows a side view of a stent from the kit in accordance with FIG. 1; and

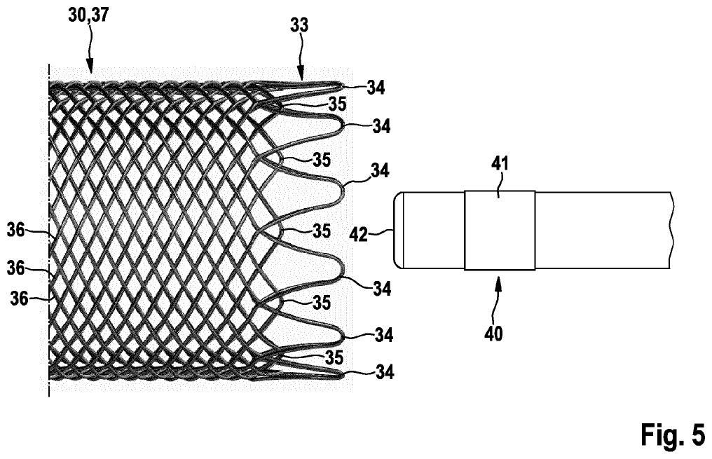

FIG. 5 shows a side view of the stent of FIG. 4 and a catheter tip before reintroducing the catheter tip into the stent.

FIG. 1 shows a kit in accordance with the invention, wherein the introducer 10 is shown in longitudinal section. A transport wire 20 is disposed in the introducer 10. The transport wire comprises a guide section 27 and a transport section 26. A stent 30 is positioned in the transport section. The transport section extends distally beyond the stent and forms a distal tip 21 there.

The distal tip 21 is a part of the transport section 26, which has a smaller cross sectional diameter than the proximally disposed guide section 27. The abutment surface 28 is provided for the transition between the transport section 26 and the guide section 27. The abutment surface extends substantially annularly around the transport section 26. A proximal radiographic marker 23 of the transport wire 20 is directly adjacent to the abutment surface 28. The radiographic marker 23 may also form the abutment surface.

The transport wire 20 has a gripping section 25 at the proximal transport wire end 24. The gripping section 25 comprises ribbing so that a surgeon can get a good grip on the transport wire.

As can be seen as well in FIG. 1, only a distal section of the transport wire 20 with the stent 30 is disposed within the introducer 10. The introducer 10 may have a plastic tube or a plastic shaft. Preferably, the introducer is transparent.

Not shown, but preferably present, is a connecting element at the distal end of the introducer, wherein a direct connection can be produced between the introducer and a catheter with the connecting element. Preferably, a catheter is used which has an internal diameter which corresponds to the internal diameter of the introducer. This ensures that the stent in the compressed state can be transferred from the introducer 10 into a catheter 40.

FIG. 1 also shows that the proximal stent end 33 is preferably disposed between two radiographic markers 22, 23 of the transport wire 20. In this regard, the distal radiographic marker 22 may be configured in a manner such that it has radially outwardly projecting protrusions, which engage in the meshwork of the stent 30. In this manner, the stent is not solely guided distally via the transport wire 20 to a treatment site; rather, it is also possible to pull a partially released stent 30 back into the catheter 40 again by means of the transport wire 20.

Feeding the stent 30 through the catheter 40 is shown in FIG. 2. It can be seen therein that the catheter, which is shown in longitudinal sectional view, has a distal catheter marker 41 at a distal end. In principle, a plurality of distal catheter markers 41 may also be provided. Preferably, the catheter marker 41 comprises a radiopaque material. Because of the position of the radiographic markers 22, 23 and of the catheter marker 41, a surgeon can readily detect the exact position of the stent 30 and adjust it if necessary.

FIG. 3 shows a blood vessel 50 in longitudinal section in which an aneurysm 51 has formed. The medical kit is used to extensively shield the aneurysm from a flow of blood. Here, the stent 30 is guided to the treatment site via a catheter 40 by means of the transport wire 20. Next, the stent 30 is released from the catheter 40. The stent thereupon expands and bears against the vessel wall. The stent therefore bridges over a neck of an aneurysm 51 and causes the flow of blood into the aneurysm 51 to be reduced. This leads to the formation of thrombi in the aneurysm 51, so that it essentially atrophies.

FIG. 4 shows a side view of a stent 30. However, for better comprehension, a rear half of the stent 30 is hidden. In the expanded state, the stent 30 preferably has an external diameter of between 2.5 mm and 3.5 mm.

The stent 30 has a meshwork 37, which is formed from wires 36. The wires 36 preferably each comprise a core produced from a radiopaque material and a sheath produced from a shape memory material. Preferably, the stent 30 has 48 wires. The wires 36 are open at the distal stent end 31 or form open wire ends 32. However, at the proximal stent end 33, each wire 36 turns around to form loops 34, 35. In addition, FIG. 4 shows that the stent 30 preferably flares conically at the proximal stent end 33. In this manner, it is additionally ensured that the proximal stent end 33 can expand properly and sits closely against a vessel wall.

At the proximal stent end 33, the stent 30 has first loops 34 and second loops 35. The first loops 34 protrude beyond the second loops 35 in the axial direction. In other words, the first loops 34 form long loops, whereas the second loops 35 form short loops. The first loops 34 and the second loops 35 alternate in a regular manner. Thus, in the circumferential direction, each first loop 34 is followed by a second loop 35, whereupon another first loop 34 and then a second loop 35 follows. As a result, every second loop in the circumferential direction is a first loop 34 or long loop.

FIG. 5 particularly clearly shows the operating principle of the invention. In the medical kit described herein, the stent 30 is therefore disposed in the introducer 10 in a manner such that the loops 34, 35 of the stent 30 are disposed at the proximal stent end 33. After the stent 30 has been released from the catheter 40, then, the loops 34, 35 are orientated in the direction of the catheter tip 42. This can be seen in FIG. 5.

It should be noted here that in FIG. 5, the conical widening of the proximal stent end 33 cannot be seen because this shows the shape of the stent 30 in the implanted state. While the non-operational state of the stent 30 is shown in FIG. 4, i.e. the stent is completely expanded without the external application of force, FIG. 5 shows the state which is taken up by the stent 30 in a blood vessel. In this regard, the blood vessel wall acts as a resistance against widening of the stent 30, so that the proximal stent end 33 does not widen conically. Moreover, the proximal stent end 33 lies closely against a vessel wall.

FIG. 5 clearly shows that because the loops 34, 35 are at the proximal stent end 33, the risk of the catheter 40 becoming caught on the meshwork 37 when the catheter 40 is moved in the distal direction is significantly reduced. If it passes along the vessel wall onto the meshwork, the catheter 40 or the catheter tip 42 slides off the loops 34, 35 and in this manner it is guided into the interior of the stent.

It should be noted that in all of the accompanying drawings, elements on the left hand side of the sheet are disposed “distally” and elements on the right hand side of the sheet are disposed “proximally”.

### LIST OF REFERENCE NUMERALS

10 introducer
20 transport wire
21 distal tip
22 distal radiographic marker
23 proximal radiographic marker
24 proximal transport wire end
25 gripping section
26 transport section
27 guide section
28 abutment surface
29 distal end of guide section 27
30 stent
31 distal stent end
32 open wire end
33 proximal stent end
34 first loop
35 second loop
36 wire
37 meshwork
40 catheter
41 distal catheter marker
42 catheter tip
50 blood vessel
51 aneurysm

1-14. (canceled)

15. A medical kit for treatment of one or more diseases of a blood vessel comprising:
a tubular introducer;
a transport wire having a distal tip; and
a stent including a meshwork of multiple wires configured and arranged to form loops at a proximal stent end and open wire ends at a distal stent end, wherein the stent is disposed on the transport wire and further disposed inside the tubular introducer, and wherein the distal stent end is positioned closer to the distal tip of the transport wire than the proximal stent end.

16. The medical kit according to claim 15, wherein the proximal stent end is disposed between a proximal radiographic marker and a distal radiographic marker of the transport wire.

17. The medical kit according to claim 16, wherein the transport wire has a transport section and a guide section having a diameter larger than a diameter of the transport section, and wherein the guide section has an abutment surface on which the loops of the proximal stent end bear in an axial direction.

18. The medical kit according to claim 17, wherein the proximal radiographic marker of the transport wire is disposed at a distal end of the guide section.

19. The medical kit according to claim 17, wherein the proximal radiographic marker directly borders the abutment surface and forms the abutment surface.

20. The medical kit according to claim 17, wherein the transport section of the transport wire extends completely through the stent.

21. The medical kit according to claim 17, wherein the distal radiographic marker of the transport wire is disposed in the transport section.

22. The medical kit according claim 15, wherein the transport wire protrudes proximally out of the introducer and has a proximal gripping section with ribbing.

23. The medical kit according to claim 15, wherein a distal end of the introducer has a connector for connection to a catheter.

24. The medical kit according to claim 15, wherein the loops comprise first loops and second loops disposed at the proximal stent end, and wherein the first loops protrude beyond the second loops in an axial direction.

25. The medical kit according to claim 24, wherein a same number of the first loops and the second loops are provided at the proximal stent end, and wherein the first loops and the second loops are disposed in alternation in a circumferential direction.

26. The medical kit according to claim 15, wherein the wires of the meshwork have a core including a radiopaque material and a sheath including a shape memory material.

27. The medical kit according to claim 26, wherein at least 10% by volume of each wire of the meshwork is formed by the core.

28. The medical kit according to claim 15, wherein the stent has no additional radiographic markers and exclusively consists of the multiple wires.

29. A medical kit for treatment of one or more diseases of a blood vessel comprising:
a tubular introducer;
a transport wire having a distal tip, wherein the transport wire has a transport section and a guide section having a diameter larger than a diameter of the transport section; and
a stent including a meshwork of multiple wires configured and arranged to form loops at a proximal stent end and open wire ends at a distal stent end, wherein the stent is disposed on the transport wire and further disposed inside the tubular introducer, wherein the distal stent end is positioned closer to the distal tip of the transport wire than the proximal stent end, wherein the guide section has an abutment surface on which the loops of the proximal stent end bear in an axial direction, wherein the loops comprise first and second loops disposed at the proximal stent end, and wherein the first loops protrude beyond the second loops in an axial direction.
